(19) United States
(12) Patent Application Publication
(10) Pub. No.: US 2024/0307652 A1
(43) Pub. Date: Sep. 19, 2024

(54) DREAM DIRECTING SYSTEM AND METHOD FOR USING THE SAME

(71) Applicant: Kenneth Rajan Leslie, North Kentville (CA)

(72) Inventor: Kenneth Rajan Leslie, North Kentville (CA)

(21) Appl. No.: 18/183,312

(22) Filed: Mar. 14, 2023

(51) Int. Cl. A61M 21/02; A61B 5/00; G06F 3/01

(52) U.S. Cl. CPC A61M 21/02; A61B 5/4812; A61B 5/6803; G06F 3/013; A61M 2021/0016; A61M 2021/0022; A61M 2021/0027; A61M 2021/005; A61M 2021/0072

(57) ABSTRACT

The disclosure is a computer-implemented method for detecting and influencing dreaming. In one embodiment, the user wears a smartwatch paired with a smartphone. The application monitors the sensors to detect REM sleep in real-time. The application allows users to set their dream incubation via the selection of an existing dream script or the coding of a new one. The dream script consists of instructions to play a series of cues: light, sound, vibration, etc. separated by user-specified time delays. The user selects a wake-up window, during which dream script playback can occur, and the dream script is previewed just before sleep. The application waits until the appropriate time and monitors for REM sleep. When REM sleep is detected , the dream script plays back. Some of the stimuli are incorporated into the dream, reminding the dreamer of their intention while also biasing the dream in the desired direction.

### CROSS-REFERENCE TO RELATED APPLICATIONS

PPA 63/319,919

### PRIOR ART

Dreaming likely evolved as a way to wire-up the visual cortex during development (Marks et al., 1995), while adult dreaming may serve many different roles, including as a threat simulator to allow for the practice of motor skills relevant to survival during sleep (Revonsuo, 2000). Although the visual cortex is very active during REM sleep/dreaming, the prefrontal cortex is normally deactivated during REM sleep (Hobson & Pace-Schott, 2002) and so the dreamer does not question the reality of the dream. Lucid dreaming occurs when the dreamer realizes that they are dreaming, and can in turn influence the dream (LaBerge et al., 1986).

Lucid dreaming is a learnable skill but it requires dedication and practice: it can often take learners 30 nights of trying before they have their first lucid dream. Even experienced lucid dreamers only have lucid dreams infrequently. For a review of lucid dream induction techniques, see Stumbrys et al. (2012). The desire for easier lucid dreaming has motivated attempts at technology aimed at inducing lucid dreams, including a variety of sleep masks and Electroencephalography (EEG) headsets. One of the first devices was the DreamLight, developed by LaBerge & Lind in 1987, which used a sleep mask with an IR eye movement sensor to detect REM sleep, which would then trigger light flashes on the eyes (LaBerge, 1988; LaBerge & Levitan, 1995). This device had some success at inducing lucid dreams. The flashing light would incorporate into the dream, and the user had to remember that the flashing light was a cue that they were dreaming. If they did, this could induce a lucid dream. A patent was filed in 1991 and granted in 1996 (U.S. Pat. No. 5,507,716), and the device was eventually released as a smart sleep mask called the NovaDreamer, which was available from 1993-2004. Although the NovaDreamer generated a lot of excitement and interest, it was never commercially successful. The original price point was expensive at $2500 USD and there was the challenge of educating customers about the benefits of lucid dreaming. The device was used at lucid dreaming events hosted by LaBerge's Lucidity Institute. The Lucidity Institute announced in 2009 a follow-up to NovaDreamer, but it was never deployed.

Since the NovaDreamer, there have been other attempts at creating lucid dream induction devices, including a number of failed crowdfunding campaigns. For example the Aurora was an EEG headband that ran a crowdfunding campaign in 2013, and although they raised almost $240,000 USD, the product never saw the light of day. Many other products have been attempted in the space, e.g., REM-Dreamer, SmartLucider, Zmax, iBand, iBand+, Neuroon, Aladdin, Lucid Catcher, but none of them have been commercially successful, and many have never been released (e.g., iBand+, Aladdin). For a review see Mota-Rolim et al. (2019). Another crowdfunding campaign in 2018-2019 raised over $300,000 USD to create the Instadreamer, which was supposed to be a wearable that could detect REM sleep and then vibrate to alert the dreamer they were dreaming (see: https://www.indiegogo.com/projects/instadreamer-take-control-of-your-dreams/#/). However, the device was never released and the project failed in 2021. The designers blamed their failure on semiconductor shortages caused by Covid-19.

There is a lucid dream induction mask currently on the market and available for $100 USD called the Remee (https://sleepwithremee.com/). This device consists of a basic sleep mask that randomly flashes red LED lights onto the eyes of the user throughout the night. Because the stimuli are not targeted, the Remee is not very effective at inducing lucid dreams.

In 2012, Kamal et al. developed a prototype device called the DreamThrower, that detected REM sleep and was able to present light flashes and simple environmental sound stimuli (rain, ocean sounds, etc.). The DreamThrower implemented REM sleep detection using an eye mask with IR emitter and sensor. The device was controlled by an onboard Arduino board. The stimuli were triggered by the rapid eye movements associated with REM sleep. The authors proposed an online socialization of dreams component that was never implemented, where users could share their dream experiences by self-reporting their dreams, and then “throw” the stimuli to online DreamThrower friends. This is similar to the concept of Dream Scripts that are developed in detail in this disclosure.

In 2014 there was a significant paper published in Nature Neuroscience (Voss et al., 2014) that claimed that transcranial AC stimulation (tACs) in the gamma band (40 Hz) applied to the forehead during REM sleep could induce lucid dreams. This finding generated a lot of excitement, and led to the development of the Alladin lucid dream induction device (https://aladdindreamer.com/). This device purported to use EEG to detect REM sleep and then 40 Hz tACs to induce lucid dreams. However, development of Alladin stopped in 2018. Moreover, no one has been able to replicate the original Voss et al. finding, and now many in the lucid dream space consider the finding to be an artifact.

There is currently a digital therapeutic called Nightware (see: https://nightware.com/) aimed at nightmare sufferers. Nightwear is built on the Apple Watch and iPhone platform, and is designed to detect nightmares through the detection of a stress response during sleep (Davenport & Werner, 2023). The Nightware app then vibrates the Apple Watch in an attempt to calm the nightmare sufferer and alert them that it is just a dream. Nightwear has patents for their device (U.S. Pat. No. 10,765,831, U.S. Pat. No. 11,284,834, U.S. Pat. No. 11,471,644, U.S. patent application Ser. No. 17/948,561), and their product is FDA approved and only available by prescription. The disclosed Dream Directing System is similar to Nightware, in that one embodiment involves a smartwatch (Apple Watch as a non-limiting example) paired with a smartphone. However, Nightwear is specifically focused on nightmare detection via the detection of stress events during sleep, and not the detection of REM sleep in particular. Nightwear also only features vibration of the wearable for stimuli, while the disclosed Dream Directing System uses complex multimodal stimuli to assist the dreamer in realizing an incubated dream.

A patent was issued to Imran & Harris from Incube Laboratories (U.S. Pat. Nos. 9,620,027; 10,019,908; 10,685,577) around a sleep headband for detecting dreaming using EEG and then playing auditory stimuli for the purpose of influencing the dream. In particular, they seem to be interested in playing tones related to the EEG signal. No device has been made available to the public by Incube Laboratories.

The Dormio is an experimental device that is worn on the hand that can be used to influence hypnogogic imagery at sleep onset through the playback of an audio cue (Horowitz et al., 2020). Note that this device does not target REM sleep, only sleep onset.

There has been research on dream incorporation of external stimuli into non-lucid dreams, including light, sound, smell, touch, pressure, and sleep rocking (Leslie & Ogilvie, 1996; Solomonova & Carr, 2019). For a review of these different stimuli regimes and their potential for dream engineering, see Carr et al. (2020).

An important breakthrough came in 2021 when Konkoly et al. showed that they were able to play stimuli to dreamers in the lab, including light, sound, and touch, and that dreamers could experience these stimuli as being incorporated into the dream. Moreover, dreamers were able to signal to the experimenter with eye movements via Electrooculogram (EOG) that they had experienced the stimuli, including answering simple math questions using eye movements. This is one of the best examples of a dreamer communicating in real-time with an experimenter. And, the finding validates that external stimuli can be incorporated into the dream, and that this can induce more self-reflective thinking, and that the dreamer can later report the presence of the stimuli during the subsequent dream report after being woken-up. The disclosed Dream Directing System seeks to take this finding from the lab to the user's home sleep space, and put the power in the hands of the user to select their own stimuli for dream incorporation.

The disclosed Dream Directing System provides users with a holistic new way to incubate, interact with, and capture their dreams. Adults spend approximately 90 minutes each night in REM sleep hallucinating non-sensensical and disturbing events that are rapidly forgotten upon awakening. The disclosed Dream Directing System allows users to bias their dreams towards a desired intention via playback of user-selected cues. The system is also able to wake users from a directed dream in order to capture a dream report at the optimal time, i.e., upon waking from a REM sleep period. This wake-up alarm could be an audio dream ad (Marlan, 2023). Dream ads may be controversial with some users, and positioning the ad as a wake-up alarm would improve transparency, as well as memory for the ad. Moreover, the Dream Directing System can be used to target the last REM sleep period of the night, which is the least disruptive to sleep. The system can transcribe the dream report using voice-to-text and convert it into images and short form video using AI. Dream directing can help support a new culture of dreaming that allows humans to use the dream simulator built by evolution to simulate user-desired experiences. This technology will be of interest to lucid dreamers, those who want to remember their dreams, nightmare sufferers who want to flip-the-script on bad dreams, as well as people interested in dream affirmations, practicing skills during sleep (as the ultimate form of visualization) and artists (art, music, film, etc.) seeking dream inspiration.

### ACADEMIC REFERENCES

Carr, M., Haar, A., Amores, J., Lopes, P., Bernal, G., Vega, T., . . . & Maes, P. (2020). Dream engineering: Simulating worlds through sensory stimulation. Consciousness and cognition, 83, 102955.

Davenport, N. D., & Werner, J. K. (2023). A randomized sham-controlled clinical trial of a novel wearable intervention for trauma-related nightmares in military veterans. Journal of Clinical Sleep Medicine, 19(2), 361-369.

Hobson, J. A. (1990). Sleep and dreaming. The Journal of neuroscience, 10(2), 371.

Hobson, J. A., & Pace-Schott, E. F. (2002). The cognitive neuroscience of sleep: neuronal systems, consciousness and learning. Nature Reviews Neuroscience, 3(9), 679-693.

Horowitz, A. H., Cunningham, T. J., Maes, P., & Stickgold, R. (2020). Dormio: A targeted dream incubation device. Consciousness and cognition, 83, 102938.

Kamal, N., Al Hajri, A., & Fels, S. (2012). DreamThrower: An audio/visual display for influencing dreams. Entertainment Computing, 3(4), 121-128.

Konkoly, K. R., Appel, K., Chabani, E., Mangiaruga, A., Gott, J., Mallett, R., . . . & Paller, K. A. (2021). Real-time dialogue between experimenters and dreamers during REM sleep. Current Biology, 31(7), 1417-1427.

LaBerge, S., Levitan, L., & Dement, W. C. (1986). Lucid dreaming: Physiological correlates of consciousness during REM sleep. The journal of mind and behavior, 251-258.

LaBerge, S., & Lind, R. (1987). Varieties of Experience from Light Induced Lucid Dreams. Lucidity Letter, 6(2).

LaBerge, S. (1988). Induction of lucid dreams including the use of the Dreamlight. Lucidity Letter, 7(2).

LaBerge, S., & Levitan, L. (1995). Validity established of DreamLight cues for eliciting lucid dreaming. Dreaming, 5(3), 159-168. https://doi.org/10.1037/h0094432

Leslie, K., & Ogilvie, R. (1996). Vestibular dreams: The effect of rocking on dream mentation. Dreaming, 6(1), 1.

Marks, G. A., Shaffery, J. P., Oksenberg, A., Speciale, S. G., & Roffwarg, H. P. (1995). A functional role for REM sleep in brain maturation. Behavioural brain research, 69(1-2), 1-11.

Marlan, D. (2023). The Nightmare of Dream Advertising. Available at SSRN 4361477.

Mota-Rolim, S. A., Pavlou, A., Nascimento, G. C., Fontenele-Araujo, J., & Ribeiro, S. (2019). Portable devices to induce lucid dreams—are they reliable ?. Frontiers in neuroscience, 428.

Revonsuo, A. (2000). The reinterpretation of dreams: An evolutionary hypothesis of the function of dreaming. Behavioral and brain sciences, 23(6), 877-901.

Stumbrys, T., Erlacher, D., Schädlich, M., & Schredl, M. (2012). Induction of lucid dreams: A systematic review of evidence. Consciousness and cognition, 21(3), 1456-1475.

Solomonova, E., & Carr, M. (2019). Incorporation of external stimuli into dream content. Dreams: understanding biology, psychology, and culture, 1.

Voss, U., Holzmann, R., Hobson, A., Paulus, W., Koppehele-Gossel, J., Klimke, A., & Nitsche, M. A. (2014). Induction of self awareness in dreams through frontal low current stimulation of gamma activity. Nature neuroscience, 17(6), 810-812.

### Patents in the Space—Prior Art

LaBerge, S. P., Rich, R. S., Wright, D. K. and Kottke, D. G., Lucidity Inst Inc, 1996. Equipment and methods used to induce lucid dreams in sleeping persons. U.S. Pat. No. 5,507,716.

Raynie, A. D., Rodriguez, R. G., Forister, G. L. and Crawford, A. B., Dream Weaver JV, 1996. Dream state teaching machine. U.S. Pat. No. 5,551,879.

Lemmon, T., Lemmon Tracy Catherine, 2004. Device for recording dream recollections. U.S. patent application Ser. No. 10/810,953.—Abandoned

Kubey, A. and Davidson, A., 2011. REM-sleep directed visual alarm system and method. U.S. Pat. No. 7,956,756.

Hunter, R. A., 2014. Dream Sharing and Visualization Platform. U.S. patent application Ser. No. 14/022,220.

Schoonover, D. C. and Smiley, A. H., 2016. Dream Enhancement Apparatus and Method. U.S. patent application Ser. No. 15/203,503.

Imran, M. and Harris, J. M., Incube Laboratories LLC, 2017. Systems and methods for delivering sensory input during a dream state. U.S. Pat. No. 9,620,027.

Schoonover, D. C. and Smiley, A. H., 2018. Dream enhancement apparatus and method. U.S. Pat. No. 10,052,452.

Imran, M. and Harris, J. M., Incube Laboratories LLC, 2018. Systems and methods for delivering sensory input during a dream state. U.S. Pat. No. 10,019,908.

Weiss, C., Shambroom, J. R., Roth, T. and Wong-Simpkins, C., Aladdin Dreamer Inc, 2019. Lucid dream stimulator, systems, and related methods. U.S. Pat. No. 10,300,240.

Imran, M. and Harris, J. M., Incube Laboratories LLC, 2019. Systems and methods for delivering sensory input during a dream state. U.S. Pat. No. 10,242,585.

Imran, M. and Harris, J. M., Incube Laboratories LLC, 2020. Systems and methods for delivering sensory input during a dream state. U.S. Pat. No. 10,685,577.

Skluzacek, T., Nightware Inc, 2020. Traumatic nightmare detection and intervention. U.S. Pat. No. 10,765,831.

Milbert, R. and Skluzacek, T., Nightware Inc, 2022. Systems and methods for automated stress monitoring and intervention. U.S. Pat. No. 11,284,834

Skluzacek, T., Nightware Inc, 2022. Traumatic nightmare detection and intervention. U.S. Pat. No. 11,471,644.

Skluzacek, T., NIGHTWARE, INC., 2023. TRAUMATIC NIGHTMARE DETECTION AND INTERVENTION. U.S. patent application Ser. No. 17/948,561.

### BACKGROUND OF THE INVENTION

The present disclosure relates to dreaming, and the use of technology to detect REM sleep, which is associated with dreaming, and to in turn influence the content of a dream through the dream incorporation of any number of user-selected cues played back during REM sleep, as well as the optimized capture of dream reports using voice-to-text technology and AI tools for dream visualization. The disclosed system leverages intention, association, and suggestion to allow the user to realize an incubated dream in a process called Dream Directing.

### SUMMARY OF THE INVENTION

The following presents a simplified summary of one or more embodiments of the present disclosure to provide a basic understanding of such embodiments. The summary is not an extensive overview of all contemplated embodiments, and is intended to neither identify key or critical elements of all embodiments, nor delineate the scope of any or all embodiments.

The disclosed Dream Directing System detects REM sleep based on signals from a sensor array. One embodiment of this sensor array is a smartwatch (with PPG heart rate and accelerometer sensors), where REM sleep is detected using a machine learning model. Other embodiments are possible, including sensor arrays based around a REM sleep detection mask (e.g., using eye movement detection via EOG or infrared sensor, and head movement via accelerometer), EEG headset, or webcam with microphone (e.g., Google Nest as a non-limiting example). Each of these embodiments would involve specific REM sleep detection machine learning models based around their unique sensor systems and calibrated against home or lab-based polysomnography measures or other techniques.

The system uses a computer system as an interface, to specify the dream script. In one embodiment, the Dream Directing System is implemented as a smartphone app that includes a dream script editor, a way to select from a menu of dream scripts, a way to preview the dream script just before sleep onset, as well as a way for the user to specify a wake-up window during which the system will detect for REM sleep and then playback the dream script.

Once REM sleep/dreaming is detected using the sensor array, the system then plays back complex dream scripts that are associated with a specific dream incubation. The Dream Directing System then wakes the user to collect a verbal dream report, which is then converted into text using voice-to-text technology. This report can then be converted into images and short form video for sharing on social media.

The Dream Directing System is designed to bias the user's dream in a desired direction, towards the incubated dream. The system can direct non-lucid dreams towards a desired experience as well as induce lucid dreams.

### BRIEF DESCRIPTION OF DRAWINGS

While the specification concludes with claims particularly pointing out and distinctly claiming the subject matter that is regarded as forming the various embodiments of the present disclosure, it is believed that the invention will be better understood from the following description taken in conjunction with the accompanying figures:

FIG. 1: System Diagram of one embodiment using smartwatch sensor array and smartphone.

FIG. 2: Flow chart of use of Application.

FIG. 3: Exploration of different embodiments.

FIG. 4a-f: Screenshots from current Alpha App implementation.

FIG. 5a-c: Drawings of different embodiments of the Dream Directing System.

### DETAILED DESCRIPTION OF THE INVENTION

The Dream Directing System comprises a system, regulated by a general processor (e.g., iPhone as a non-limiting example), that is implemented in a sleep space (i.e., the bedroom, or any other space where a user may experience sleep), that has access to both sensors (including but not limited to heart rate monitor, movement monitor, eye movement detection system, skin temperature, EEG, EOG, Electromyography (EMG), video camera, webcam, microphone, etc.) and various means of stimulation (including but not limited to light, sound, vibration, motion, electrical stimulation, scent, etc.). Dream directing begins with an intention to have a specific dream. Once this intention is set, the device is programmed with an appropriate script (or series of instructions) to provide one or more stimuli including but not limited to environmental and physical stimuli. In some embodiments, the stimuli may include the sound of waves and a rocking motion of the bed, for example. The script includes instructions for different stimuli to be played back during Rapid Eye Movement (REM) sleep, which is associated with dreaming. This could include, by way of a non-limiting example, flashing a red light on the eyes, playing an auditory stimuli, e.g., “you are dreaming, this is a dream”, or even air perfusion with a scent (e.g., lavender) as well as possible activation of a motion bed (which rocks the dreamer to provide vestibular stimulation). Before the user goes to sleep, the user can preview the dream script, i.e., play the dream script to familiarize themselves with the script cues and help set their intention to have the directed dream. The preview helps the user associate the different cues with different parts of the dream script, which will help them remember their intention when the cues are presented during dream sleep, i.e., as a cognitive support to the dreamer.

The Dream Directing System may include a REM sleep detection system. There are several different sensory arrays and algorithms/models that can be used for REM sleep detection. Example sensor systems include but are not limited to measures of heart rate (HR) and Heart Rate Variability (HRV), head movement, body movement, as well as eye movements (via EOG, video camera, infrared sensor), brain waves via EEG, muscle tension via EMG, skin temperature and changes in breathing. REM sleep detection is accomplished by means of sensors that are processed by a REM sleep detection algorithm (implemented as a machine learning model), either at the level of the sensor system (e.g., smartwatch as a non-limiting example) or at the level of the general processor controller, which then determines in real time if the user is in REM sleep. Note, for most users, REM sleep is most abundant in the later part of the night, and so the system can be set to specifically target later REM periods, e.g., after 6 hours of sleep or during a user-defined wake-up window. However, such criteria can be customized for each user, and some users may prefer to target every REM period detected.

Once REM sleep is detected, the Dream Directing System plays back the dream script. Said another way, the general processor will execute the series of instructions (i.e., script playback). Many different stimuli can be used, including:
Auditory, including music, speech (and user recorded speech), affirmations
Light flashes, different colored light
Movement, including rocking of any kind
Scent
Vibration of smartwatch, sleep mask or bed insert
Electrical stimulation of the wrist or scalp (e.g., tACs, tDCs, etc.)
Magnetic stimulation to activate brain tissue associated with sensory processing
Temperature stimulation
Mechanical stimulation, including pressure and light touch

The stimuli from the dream script are played back in the sequence specified by the script during REM sleep, and some of these stimuli will become incorporated into the dream by the process of dream incorporation. Said another way, normally the dream is created at the level of the cortex, from Ponto-Geniculo-Occipital (PGO) waves that emanate from the brainstem, project to the thalamus, and then are sent on to the cortex where they are interpreted by the cortex to create the dream experience (Hobson, 1990). However, thalamic blockade is incomplete during REM sleep, and some external sensory stimuli are able to reach the thalamus and get mixed with existing PGO waves at the level of the thalamus, and then get projected to the cortex, where they are interpreted by the cortex as the dream (i.e., a simulation of reality), allowing for dream incorporation of external stimuli during REM sleep (Konkoly et al., 2021).

Within the dream, the dreamer senses these familiar cues (i.e., the dream script cues), which have been incorporated into the dream in some form. These stimuli begin to bias the dream content towards the desired dream experience. This can lead to the realization of the incubated dream. This can also lead to the realization that one is dreaming, i.e., lucid dream induction. For dream directing to be successful, you need to play the right stimuli at the right time (REM sleep) and at the right level of intensity (strong enough to be perceived, but not so strong as to wake the user). Although lucid dream induction is a desirable by-product of the Dream Directing System, this is not the core objective. The goal is to have a dream experience that is similar to what was incubated/intended.

Even when dreamers do become lucid, they often lose their lucidity during the dream, and the dreamer forgets to follow-up on their original intentions. The Dream Directing System, by means of the dream script playback during REM sleep, helps remind the dreamer of their intentions, while also biasing the dream towards the desired direction. The system combines the powers of intention, association and suggestion for maximal effect. The dream script will similarly help lucid dreamers remember to act on their goals during a lucid dream.

In some embodiments, the Dream Directing System can include a sleep/dream mask that is able to detect eye and head movements, and also able to flash light cues onto the user's closed eyes, as well as play back audio cues and vibrate. This mask can help detect when REM sleep has started by detecting eye movements, and it can also act as a user interface for a lucid dreamer, acting as a “dream joystick.” If the dreamer becomes lucid, they can signal with their eyes that they are lucid (e.g., by looking side-to-side 3 times). They could even use their eye movements as a simple controller within the dream, for example, looking down for 3 seconds could toggle specific cues on-and-off, e.g., a motion bed could be turned on and off, looking right for 3 seconds could trigger a light stimuli, looking left could replay the last auditory cue, and looking up could restart the dream script. Other instructions may be programmed into the REM sleep detection mask interface as desired.

In some embodiments, other sensory systems could be used to detect REM sleep. An EEG headset with EOG and EMG is the gold standard for detecting REM sleep. Similarly, it may be possible to detect REM sleep using a webcam with a microphone and an appropriate machine learning algorithm (e.g., Google Nest as a non-limiting example).

One issue with dreaming in general is that individuals often quickly forget them, because of the difference in neuromodulation between REM sleep and wake. This memory issue can be addressed by capturing a dream report upon waking using voice-to-text technology. For example, at the end of the dream script playback, a wake-up alarm is played to wake the user (e.g., an auditory cue that repeats and increases in volume, electrical stimulation, vibration, or other cue such as an auditory dream ad), and the user is prompted to give a dream report, e.g., “mentation report?”, and they proceed to give a verbal report of the dream. The dream report may be captured using a simple audio or video recorder, which may be saved in a memory device that is either integral to the processor or external to it (e.g., in a smartwatch, smartphone or in the cloud). The audio or video recording may also be converted to text for easy reference and searchability, including the addition of hashtags.

In some embodiments, an auditory dream ad can be played back as the wake-up alarm, and then a dream report is collected that could include the dream ad that was used as part of the wake-up alarm. The dream scripting software could give users a selection of auditory dreams ads to choose from for wake-up or insertion into the dream script proper.

In some embodiments, voice-to-text may be used to save the dream report in a user database. Also, in some embodiments, the system wakes up the sleeper at the end of the dream script by means of different stimuli, e.g., electrical stimulation, rocking, or sound alarm. A dream report is one step towards visualizing the dream. The Dream Directing System can include an AI that converts the text dream report into images, e.g., a cartoon with a number of panels, images, or video, including short form video. These images and video can then be shared by the user to social media.

In some embodiments, AI visualization may be done by incorporating part of the original scripting process as additional cues to the AI. During the scripting process, images of locations and activities may be used to help the dreamer in the visualization process as well as to prepare the dreamer for the dream incorporation, i.e., a form of dream incubation. After reporting the directed dream, there will be a correlation between the cues that were originally scripted and the user's dream report. The above will serve two purposes. The first is to establish a correlation metric of how close the scripted dream is to the realized one; and hence the accuracy of the Dream Directing System as a whole. The second is to aid the actual conversion of text dream reports into images, i.e., both the dream report and the dream script could be used to inform the AI generated visualization.

Using machine learning analysis of user data from many different users, the system can also help identify which dream scripts are most effective, and these scripts could then be shared with other users. In this way, dream scripts could become a valuable new media form for users to create and enjoy, and a dream script economy could be created.

Dream scripts also allow for users to explore the concept of shared dreaming by having two or more users incubate the same dream script. This could provide a fun activity for a group of people (e.g., fans of a film franchise, LARPers, etc.) and it could be especially useful for couples who are living in different cities and want to experience their loved one in the dream world.

Dream scripts could also be used to incubate sex dreams that help people who are unable to have to have sexual relations for any number of reasons (paralysis, isolation, old age, etc.).

It is also possible in some embodiments to start to visualize the dream by visualizing the behavior of the dreamer. This can be done with the REM sleep mask by recording eye movements, and then later projecting those eye movements onto a digital avatar of the user, showing the user looking around in the dream, as well as using their eye movements to control the Dream Directing System within the dream. Similarly, it is possible to use a camera to record facial expressions during REM sleep, and these can also be recorded and projected onto a digital avatar. Moreover, it is possible to record signals from the muscles near the larynx to attempt to record and decipher the user's dream speech. It is possible to train an AI system to decode silent speech if appropriate sensor information is available. Again, in one embodiment, the Dream Directing System can include software that visualizes the dream based on movements (eye movements and facial expressions as an example) from the REM sleep recording. This helps users remember the dream better, enhancing the long term benefit of the dream.

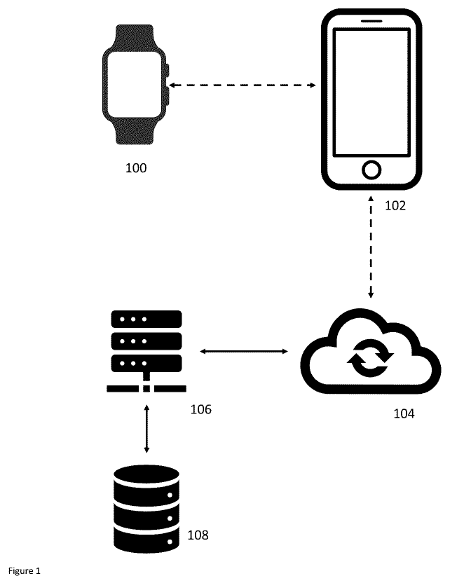

FIG. 1 is a system diagram illustrating an embodiment of the present disclosure. In it, a smartwatch 100 may connect to a smartphone 102 via Bluetooth or some other means (e.g., Wifi as a non-limiting example). The smartphone 102 may connect via a cloud network 104 to a server 106. The server 106 may connect to a database 108. The database 108 may contain data collected by the smartwatch 100 and/or the smartphone 102. One skilled in the art will recognize that many other configurations are possible. For example, instead of running the application on the smartphone 102 that communicates with the smartwatch 100, one might omit the smartphone 102 and run the application directly on the watch 100 and use a web-based interface for dream script creation and selection. Also, one might omit the network 104, server 106, and database 108 and run in off-line mode. In addition, one might replace the watch 100 with a variety of other devices capable of monitoring a person during sleep and detecting REM sleep in real-time, as well as other systems for providing external stimuli (cue playback) to the sleeper. Systems that could replace the smartwatch 100 include: REM sleep detection mask, EEG headset, webcam with microphone, armband, chest band, bed monitor, patch, etc. For stimuli playback, one can use a range of devices/peripherals, including playback of stimuli (light and sound) from a smartphone 102, as well as vibration of the smartwatch 100, as well as other effectors, e.g., a rocking bed, transcranial AC stimulation, transcranial DC stimulation, scent perfusion, etc. In addition, one might combine these devices in a variety of ways.

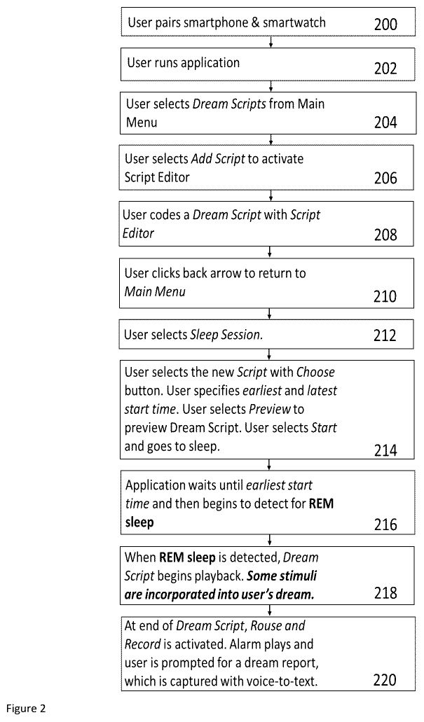

FIG. 2 is a flowchart of an embodiment of the present disclosure. In it, a user may begin at step 200 by pairing a smartphone 102 and a smartwatch 100. At step 202, the user may run the application on a smartphone 102. At step 204, the user starts the application and navigates to the Dream Scripts menu. From there, they can either select an existing script or select to add a new script 206. This activates the Script Editor 208, where the Dream Script is built out of the selection of different stimuli (light, sound, vibrate, etc.) interspersed with user-specified delays (e.g., 5 seconds, 10 seconds, etc.) and a Rouse and Record option (e.g., alarm, dream ad, long vibration, electrical stimulation of the skin, etc.). From there, the user returns to the Main Menu 210 and selects Sleep Session 212. From the Sleep Session screen, the user selects the Dream Script they want to use, e.g., the new Dream Script coded above 208. The user specifies a Wake-up Window (i.e., earliest and latest start-time) 214 and the user Previews the Dream Script (i.e., plays back the Dream Script either in real-time or accelerated without delays), and then starts the Sleep Session and goes to sleep. The system then waits for the appropriate time window before Dream Script playback is initiated. During this window, the system begins to detect for REM sleep/dreaming 216. Once REM sleep is detected, the system begins to play back the Dream Script and some of these stimuli are incorporated into the dream, biasing the dream in the desired direction 218. Upon reaching the end of the Dream Script, the system activates Rouse and Record and the user is woken-up with the preselected Rouse and Record cue 220, e.g., alarm sound, dream ad, vibration of wearable, electrical stimulation from wearable, etc. The system then prompts the user for a dream report, i.e., “mentation report?” and the dreamer gives a verbal report which is then captured as an audio recording and converted using voice-to-text into a text dream report. This text dream report is named and entered into the Digital Dream Journal and can later be converted into images and short form video using AI.

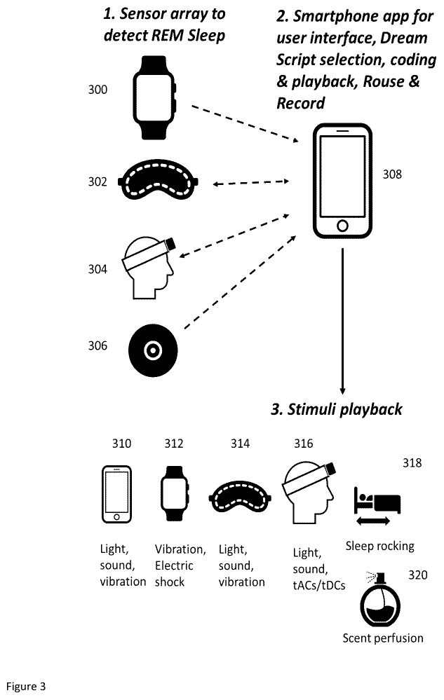

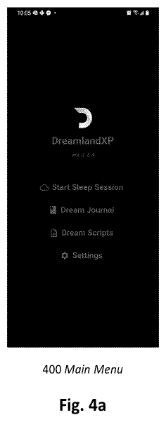

FIG. 3 illustrates that the Dream Directing System can have many embodiments based around the combination of a 1. a sensor array 300-306, 2. a General Processor, shown here as a smartphone 308, as a non-limiting example, and 3. stimuli playback via any number of devices 310-320. A variety of different sensor arrays can be used to detect when the user is in REM sleep, including a smartwatch 300 using HR and accelerometer among other possible sensors, as well as a REM sleep detection mask 302 capable of detecting eye and head movements. Note that the REM sleep detection mask could also act as a “dream joystick” by allowing a dreamer to communicate in real-time with the General Processor, e.g., the smartwatch 308, via user-specified eye movements. These specific eye movements (e.g., look left for 3 seconds then look right for 1 sec) could be detected and used to control the General Processor and app in real-time. Other REM sleep detection sensor systems include an EEG headset 304 and even a stand-off webcam with microphone 306 given appropriate software (e.g., Google Nest as a non-limiting example). Once REM sleep is detected and any other conditions are met (e.g., user specified Wake-up Window), the General Processor 308 can initiate Dream Script cue playback. Note that REM sleep detection occurs with the help of a REM sleep detection algorithm implemented as a machine learning model. The Dream Script stimuli can include different modalities, including light, sound, vibration, electrical stimulation (e.g., shock, transcranial AC stimulation, transcranial DC stimulation), sleep rocking, and scent perfusion. The playback of these different cues can be accomplished via the General Processor, e.g., the smartphone 308/310 can playback light and sound, as well as via paired devices, such as a smartwatch 312 (vibration, electric shock), REM sleep detection mask 314 (light, sound, vibration), EEG headset 316 (light, sound, transcranial AC stimulation, transcranial DC stimulation), as well as vestibular stimulation via a rocking bed 318 and olfactory stimulation via automated scent perfusion 320. Together these elements allow for directed dreaming through the detection of dreaming (REM sleep), and the playback of user-selected cues that act as a reminder of the dreamer's incubated dream, while also biasing the dream in the desired direction via dream incorporation.

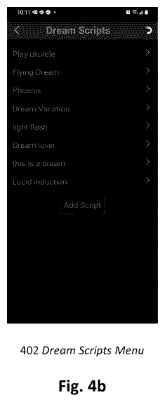

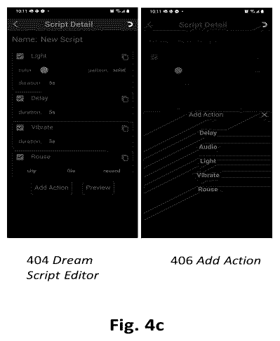

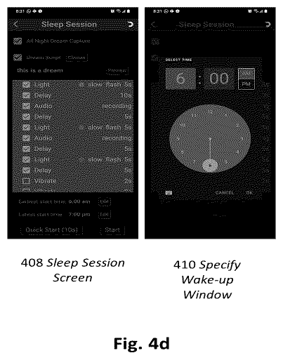

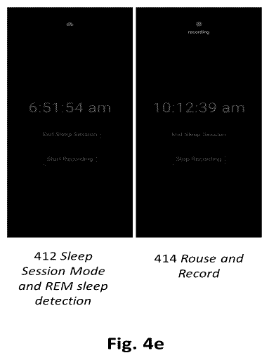

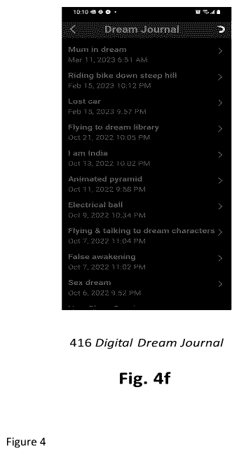

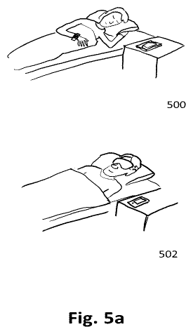

FIG. 4a-f show screenshots from an embodiment of the present disclosure. When the user opens the application they arrive at the Main Menu 400. From here, they can select Dream Scripts, which gets them to a menu of their current Dream Scripts 402. The user can elect to add a new script, which will take them to the Dream Script Editor 404, where they can build a Dream Script out of various actions, including Delay, Audio, Light, Vibrate, and Rouse and Record 406. Once the desired Dream Script is coded, the user can return to the Main Menu 400, and select Sleep Session, which brings them to the Sleep Session screen 408. From this screen, they can specify a Wake-up Window by selecting the appropriate earliest start time 410 and latest start time. The user then Previews the Dream Script from this window, which plays back the Dream Script, either at regular speed or without any delays, while the user is in the sleep space and ready to go to sleep. Once they are ready, the user presses the Start button 408. The app then goes into Sleep Session Mode 412, and waits for the appropriate time (i.e., the Wake-Up Window specified earlier 410). During this time, the system is still able to capture a wild dream report if the user wakes-up and speaks their dream. Once the Wake-up Window is entered, the system actively detects for REM sleep 412 and when REM sleep is detected, the various cues are played back. The Dream Script ends with a Rouse and Record screen 414, where the app plays a user selected alarm (e.g., user's voice, alarm sound, vibration, audio dream ad, etc.) that wakes the user. The user is then prompted for a verbal dream report, and the red light shows that the report is being recorded 414. In some embodiments, the verbal dream report is automatically converted into a text report via voice-to-text, and the dream is named and stored in a digital dream journal 416. In some embodiments, this dream journal is available on the app, as well as via a web-based interface. In some embodiments, the text dream report can be converted into images or short form video using AI.

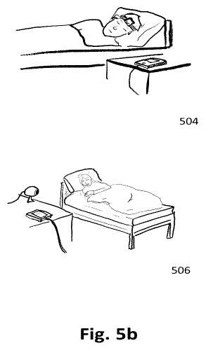

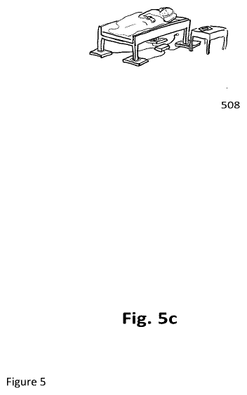

FIG. 5a-c show drawings of different embodiments of the disclosure, including a system built around a smartwatch paired with a smartphone 500, a REM sleep detection mask paired with a smartphone 502, an EEG headset paired with a smartphone 504, a webcam and microphone paired with a smartphone 506, as well as a rocking bed paired with a smartphone 508.

1. A system for directing the dreams of a user during a sleeping event, the system comprising:
at least one sensor for detecting one or more user activities during said sleeping event and for obtaining measurements of the user relating to the one or more activities detected;
a general processor for receiving the measurements, the general processor having a series of instructions, when executed allows the general processor to execute the series of instruction to perform the steps of:
determining a sleep stage of the user based on the measurements received from at least one sensor; and
triggering at least one of a physical or environmental stimulus to the user for manipulating the dream of the user during the predetermined sleep stage of the sleeping event.

2. The system according to claim 1, wherein the one or more activity of the user during the sleeping event is determined to be Rapid Eye Movement (REM) sleep.

3. The system according to claim 1, wherein at least one sensor comprises at least one of a wearable device, a REM sleep detection mask, an EEG headset, a bed insert, an infrared video camera, a webcam, or a microphone.

4. The system according to claim 1, wherein the processor comprises an interface for allowing the user to select a scenario (dream script) to be experienced during the sleeping event.

5. The system according to claim 4, wherein the scenario is a predetermined scenario (dream script) stored in a memory device accessible by the general processor.

6. The system according to claim 4, wherein the interface allows the user to enter a series of events to be experienced by the user during the sleeping event in the form of a dream script, i.e., a sequence of user-specified cues.

7. The system according to claim 1, wherein at least one of a physical or environmental stimulus includes one of a visual stimulus, an auditory stimulus, vibration of a wearable, movement of a rocking bed insert on which the user sleeps during the sleeping event, an olfactory stimulus (scent), and electrical stimulation, including electrical shock, tACs and tDCs.

8. A method for directing a user's dream during sleep using the system of claim 1, comprising determining the user's sleep stage based on measurements from at least one sensor and triggering physical or environmental stimuli to manipulate the user's dream during a predetermined sleep stage.

9. A method of using the system described in claim 8 wherein the intensity of triggered physical or environmental stimuli is adjusted to be perceived by the user without waking them up. This adjustment can be regulated by the user or managed by a machine learning model or algorithm.

10. A method of using the system described in claim 1 further comprising waking the user with a selected alarm and capturing an auditory dream report.

11. A method of using the system described in claim 1 to specify a wake-up window during which REM sleep will be detected and stimuli playback will occur, allowing users to target later REM sleep periods.

12. A method of using the system described in claim 1 and playing an auditory dream ad or user-selected dream ad that plays as part of the dream script or as a wake-up alarm.

13. A method for taking the auditory dream report in claim 10 and converting it to a text dream report using voice-to-text technology.

14. A method of using artificial intelligence (AI) to convert said text dream report in claim 13 into a series of images or video that can be shared by the user on social media.

15. A method of using the system described in claim 1 with an eye movement detection mask that can act as a “dream joystick” that allows the user to control said Dream Directing System via in-dream eye movements according to preprogrammed commands.

16. A method of using the system described in claim 1 to implement shared dreams, where two or more people are able to incubate a shared dream that is connected via the intentional playback of the same dream script (i.e., scenario or set of cues).

17. A method of using the system described in claim 1 to implement sex dreams.

18. A method of using the system described in claim 1 to gather data about eye movements, facial expressions, subthreshold vocalization and other measures to help visualize the actions of the dreamer via projection onto a digital avatar as part of dream visualization.
